(19) United States
(12) Patent Application Publication
(10) Pub. No.: US 2016/0130245 A1
(43) Pub. Date: May 12, 2016

(54) 6,8-SUBSTITUTED NARINGENIN DERIVATIVE AND USE THEREOF

(71) Applicants: Weiwei SU, Guangdong (CN); Yonggang WANG, Guangdong (CN); Zhong WU, Guangdong (CN)

(72) Inventors: Weiwei Su, Guangzhou, Guangdong (CN); Yonggang Wang, Guangzhou, Guangdong (CN); Zhong Wu, Guangzhou, Guangdong (CN)

(73) Assignee: SUN YAT-SEN UNIVERSITY, Guangzhou, Guangdong (CN)

(21) Appl. No.: 14/784,036

(22) PCT Filed: Jan. 18, 2013
(86) PCT No.: PCT/CN2013/070654

§ 371 Date: Oct. 13, 2015

(30) Foreign Application Priority Data
Dec. 19, 2012 (CN) 201210557413.0

(51) Int. Cl. C07D 311/32; A61K 31/353

(52) U.S. Cl. CPC C07D 311/32; A61K 31/353

(57) ABSTRACT

Disclosed in the present invention are a 6,8-substituted naringenin derivative as shown in formula (I):

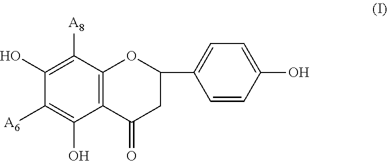

in which A₆ and A₈ in formula (I) are respectively selected from H, halogen, hydroxyl, an alkyl group with 1-3 carbon atoms, and a straight-chain alkenyl, alcohol group, aldehyde group, or carboxylic acid group containing 2-3 carbon atoms, obtained by modifying naringenin as lead compound, or a pharmaceutically acceptable salt thereof, a preparation method therefor and use thereof in preparation of drugs for improving the function of the respiratory system and relieving cough.

### TECHNICAL FIELD

The present invention relates to a naringenin derivative, and particularly a 6,8-substituted naringenin derivative and use thereof in preparation of drugs for improving the functions of the respiratory system and especially drugs for relieving cough.

### BACKGROUND ARTS

Naringenin is the aglycone of naringin, and is widely present in the raw plant tissues including cherry blossom buds, plum blossom buds, peels of citrus fruit, and pummelo peels. Naringenin and naringin both have cough relieving effect. Naringenin is chemically stable in nature and remains as such after entering human body. Naringin is a glycoside of naringenin, which is formed by attaching a disaccharide at position 7 of naringenin and converted into naringenin by removing the disaccharide through hydrolysis in the presence of gastric acid and various enzymes after entering human body. Therefore, naringenin is the main form that exerts physiological effect in human body (Kazuo Ishii, Takashi Furuta, Yasuji Kasuya. Determination of naringin and naringenin in human plasma by high-performance liquid chromatography. Journal of Chromatography B, 683(1996); 225-229). The molecular structures of naringenin and naringin are as shown below:

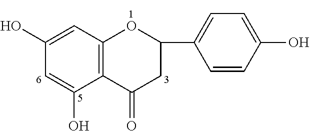 [SMILES: O=C1CC(c2ccc(O)cc2)Oc2cc(O)cc(O)c21]

### Molecular Structure of Naringenin

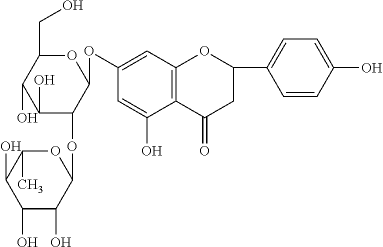

### Molecular Structure of Naringin

At present, codeine phosphate is clinically the most commonly used drug for reliving cough, which however results in addiction in clinic.

### SUMMARY OF THE INVENTION

In the present invention, a 6,8-substituted naringenin derivative is developed by the researchers of the present invention after long-term studies, by further chemically modifying naringenin as lead compound. Moreover, a method for preparing the 6,8-substituted naringenin derivative is further provided. Experiments show that the naringenin derivative and salts thereof according to the present invention are obviously effective in improving the function of the respiratory system, for example, in respect of cough relieving, facilitating expectoration, and counteracting inflammation, and especially in relieving cough, and is useful in treatment of respiratory system diseases and disorders caused by bacteria and viruses.

The 6,8-substituted naringenin derivative according to the present invention has a structure as shown in formula (I) below:

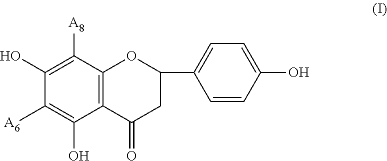

in which A₆ and A₈ are respectively selected from H, halogen, hydroxyl, an alkyl group with 1-3 carbon atoms, and a straight-chain alkenyl group, alcohol group, aldehyde group or carboxylic acid group containing 2-3 carbon atoms, provided that A₆ and A₈ preferably are not the same group.

The halogen is F, Cl, Br or I.

The salt is preferably a potassium salt or a sodium salt, and may be prepared through conventional process.

The straight-chain alkenyl group, alcohol group, aldehyde group or carboxylic acid group containing 2-3 carbon atoms is preferably straight-chain propenyl, ethanol group, acetaldehyde group, or acetic acid group.

The alkyl group with 1-3 carbon atoms is preferably methyl, ethyl or propyl. The 6,8-substituted naringenin derivative according to the present invention can be prepared through a method comprising the specific steps of:

(1) preparing the intermediate compounds 8-allylnaringenin and 6-allylnaringenin of the derivative, specifically by subjecting a known compound 7-allyloxynaringenin as starting material to Claisen rearrangement in toluene, to obtain two isomers that are 8-allylnaringenin and 6-allylnaringenin respectively;

(2) as required by the position of the modifying group to be introduced, dissolving the intermediate compound 8-allylnaringenin or 6-allylnaringenin obtained in step (1) in ethanol, adding 10% Pd/C, hydrogenating for 20 hrs under normal pressure, filtering under suction, and purifying the filtrate by silica gel column chromatography eluting with a mixed solvent (2:1) of petroleum ether and ethyl acetate, to obtain a derivative substituted with a propyl group at position 8 or position 6 respectively;

(3) as required by the position of the modifying group to be introduced, dissolving the intermediate compound 8-allylnaringenin or 6-allylnaringenin obtained in step (1) in tetrachloromethane and dimethyl formamide, halogenating with equivalent amount of N-halo succinimide, and after the reaction is complete, purifying the reaction solution by silica gel column chromatography eluting with petroleum ether:ethyl acetate 5:1, to obtain an allylnaringenin derivative substituted with halogen at position 6 or 8 respectively;

(4) as required by the position of the modifying group to be introduced, dissolving the derivative substituted with a propyl group at position 8 or 6 obtained in step (2) in tetrachloromethane and dimethyl formamide, halogenating with equivalent amount of N-halosuccinimide, and after the reaction is complete, purifying the reaction solution by silica gel column chromatography eluting with petroleum ether:ethyl acetate 5:1, to obtain a propylnaringenin derivative substituted with halogen at position 6 or 8 respectively;

(5) dissolving the intermediate compound 8-allylnaringenin or 6-allylnaringenin obtained in step (1) in dichloromethane and tetrahydrofuran, cooling the solution to −70° C. in a dry ice-acetone bath under neat nitrogen atmosphere, then introducing ozone continuously for 2 hrs until the solution becomes blue colored and no discoloration occurs in ten minutes after the introduction of ozone is complete, whereupon introducing air for 15 min, then adding a reducing agent dimethyl sulfide, gradually heating to room temperature, mixing the resulting product with silica gel and purifying by silica gel column chromatography, to obtain a naringenin derivative substituted with an aldehyde group at position 8 or 6 respectively;

(6) dissolving the allylnaringenin derivative substituted with halogen at position 6 or 8 obtained in step (3) in ethyl acetate, then adding m-chloroperoxybenzoic acid, heating to reflux for 48 hrs, filtering under suction, concentrating the filtrate, and then purifying the residue by silica gel column chromatography, to obtain an allylnaringenin derivative substituted with a hydroxyl group at position 6 or 8 respectively;

(7) dissolving the naringenin derivative substituted with an aldehyde group at position 8 obtained in step (5) in ethanol, adding 1.1 eq of amino sulfonic acid, adding 1.1 eq of sodium chlorite dropwise at freezing point, and after complete reaction, mixing the resulting product with silica gel and purifying by silica gel column chromatography, to obtain a naringenin derivative substituted with chloro at position 6 and with a carboxylic acid group at position 8, and

dissolving the naringenin derivative substituted with an aldehyde group at position 6 obtained in step (5) in ethyl acetate, adding 1.1 eq of aminosulfonic acid, adding 1.1 eq of sodium chlorite dropwise at freezing point, and after complete reaction, mixing the resulting product with silica gel and purifying by silica gel column chromatography, to obtain a naringenin derivative substituted with chloro at position 8 and with a carboxylic acid group at position 6; and

(8) dissolving the naringenin derivative substituted with an aldehyde group at position 8 obtained in step (5) in ethyl acetate, adding 1.1 eq of aminosulfonic acid, adding 1.1 eq of sodium chlorite dropwise at freezing point, and after the reaction is completed in 0.5 hr, mixing the product with silica gel and purifying by silica gel column chromatography, to obtain a naringenin derivative substituted with H at position 6 and with a carboxylic acid group at position 8.

The present invention encompasses a pharmaceutical composition which comprises the 6,8-substituted naringenin derivative as an active ingredient and a pharmaceutically acceptable carrier. Generally, the pharmaceutical composition of the present invention contains 0.1-95% by weight of the 6,8-substituted naringenin derivative according to the present invention.

The pharmaceutical composition containing the 6,8-substituted naringenin derivative according to the present invention may be prepared through a method generally known in the art. To this end, the 6,8-substituted naringenin derivative of the present invention may be combined with one or more solid or liquid excipients and/or adjuvants, if needed, to prepare drugs for human or veterinary use having suitable dosage forms for administration.

The 6,8-substituted naringenin derivative or the pharmaceutical composition containing the same according to the present invention may be administered in a unit dosage form through parental or non-parental routes, for example, oral, intramuscular, subcutaneous, nasal, transmucosal, transcutaneous, intraperitoneal, or rectal route of administration.

The 6,8-substituted naringenin derivative or the pharmaceutical composition containing the same according to the present invention may be administered through injection, including intravenous injection, intramuscular injection, hypodermic injection, intradermal injection and acupoint injection.

The dosage form for administration may be a liquid or solid dosage form. For example, the liquid dosage form may be a true solution, a colloid, a particulate, an emulsion, or a suspension dosage form. Other dosage forms include, for example, tablets, capsules, drop pills, aerosols, pills, powders, solutions, suspensions, suppositories, lyophilized powder injections, and others.

The 6,8-substituted naringenin derivative of the present invention may be prepared into an ordinary preparation, or into a sustained release preparation, a controlled release preparation, a targeting preparation, and various particulate delivery systems.

For example, where the unit dosage form is intended to be prepared into a tablet, various carriers generally known in the art may be widely used. Examples of the carriers include, for example, diluents and absorbents, such as starch, dextrin, calcium sulfate, lactose, mannitol, sucrose, sodium chloride, glucose, urea, calcium carbonate, kaolin, microcrystalline cellulose, aluminium silicate, and the like; wetting agent, such as water, glycerine, polyethylene glycol, ethanol, propanol, and the like; binders, such as starch paste, dextrin, syrup, honey, glucose solution, gum arabic, gelatin, carboxymethyl cellulose, methyl cellulose, shellac, potassium phosphate, polyvinyl pyrrolidone, and the like; disintegrants, such as dry starch, alginate, powdered agar, laminarin, sodium bicarbonate and citric acid, calcium carbonate, polyoxyethylene sorbitan fatty acid ester, sodium dodecylsulfonate, methylcellulose, ethylcellulose, and the like; disintegration inhibitors, for example, sucrose, glyceryl stearate, cacao butter, hydrogenated vegetable oil, and the like; absorption promoters, for example, quaternary ammonium salts, sodium dodecylsulfate, and the like; lubricants, for example, talc, silica, corn starch, stearate, boric acid, liquid paraffin, polyethylene glycol, and the like. The tablet may be further prepared into a coated tablet, for example, sugar coated tablet, tablet coated with films, tablet coated with enteric coating, double layered tablet, or multi-layered tablet.

For example, where the unit dosage form is intended to be prepared into a pill, various carriers generally known in the art may be widely used. Examples of the carriers include, for example, diluents and absorbents, such as glucose, lactose, starch, cacao butter, hydrogenated vegetable oil, polyvinyl pyrrolidone, kaolin, talc, and the like; binders, such as gum arabic, tragacanth, gelatin, ethanol, honey, rice paste or batter, and the like; disintegrants, such as powdered agar, dry starch, alginate, sodium dodecylsulfonate, methylcellulose, ethylcellulose, and the like.

For example, where the unit dosage form is intended to be prepared into a capsule, the 6,8-substituted naringenin derivative of the present invention as active ingredient is mixed with various carriers above, and the resulting mixture is encapsulated in a hard or soft gelatin capsule. Alternatively, the 6,8-substituted naringenin derivative of the present invention as active ingredient is prepared into a microcapsule, suspended in an aqueous medium to form a suspension, filled in a hard capsule, or prepared into an injection.

For example, the 6,8-substituted naringenin derivative of the present invention as active ingredient may be prepared into a preparation for injection, for example, a solution, a suspension, an emulsion, or a lyophilized powder injection. The preparation may be aqueous or non-aqueous, and contain one or more pharmaceutically acceptable carriers, for example, diluents, binders, lubricants, preservatives, surfactants or dispersants. For example, the diluents may be selected from the group consisting of water, ethanol, polyethylene glycol, 1,3-propylene glycol, ethoxylated isostearyl alcohol, polyoxyisostearyl alcohol, polyoxyethylene sorbitan fatty acid ester, and the like. Furthermore, in order to make the injection isotonic, a suitable amount of sodium chloride, glucose, or glycerine may be added to the preparation for injection. Further, conventional solubilizers, buffers, pH adjusting agents and others may be added as well. These adjuvants are those commonly used in the art. In addition, colorants, preservatives, fragrances, flavoring agents, sweeteners or other materials may be further added to the pharmaceutical preparation, if needed.

The dosage of the pharmaceutical composition containing the 6,8-substituted naringenin derivative according to the present invention depends on numerous factors, for example, the nature and severity of the diseases to be prevented or treated, the gender, age, body weight, disposition, and individual response of the patient or animal, route of administration, and dosage frequency etc. Therefore, the therapeutic dose in the present invention may vary over a wide range. The dosage of the pharmaceutical ingredient used in the present invention is generally known to persons of skill in the art, and may be appropriately adjusted according to the actual amount of the agent in the final preparation of the composition containing the 6,8-substituted naringenin derivative according to the present invention, to satisfy the requirement for therapeutic amount and thus accomplish the preventive or therapeutic purpose of the present invention. The daily dose of the 6,8-substituted naringenin derivative of the present invention is suitably in the range of 0.01-1000 mg/kg of body weight, and especially 1-100 mg/kg of body weight. The dose may be given in a single or several divided doses, for example, two, three or four divided doses, depending on the clinical experience of the prescribing physician and dosing regimen adopted in clinic.

It is confirmed through experiments that the 6,8-substituted naringenin derivative of the present invention are obviously effective in improving the function of the respiratory system in respect of cough relieving, facilitating expectoration, and counteracting inflammation, and most of the derivatives have a cough relieving effect superior to that of naringin or codeine. Particularly, the therapeutic efficacy of 8-n-propylnaringenin (the structure is shown in Example 1) is higher than naringenin. It can be inferred that the 6,8-substituted naringenin derivative of the present invention has a lower applicable dosage in clinic than naringin, and this class of substances are higher than naringin in therapeutic index and bioavailability. From the analysis of chemical structure-efficacy relationship of the agent, it is found that the derivative has a chemical structure similar to that of naringenin, but absolutely different from that of codeine phosphate, suffers no side effects of codeine phosphate in clinic such as addiction and resistance. Moreover, the molecular structure is simpler and more stable than naringin, and the synthesis process is simple. According, the derivative is suitable for scalable production in industry and has a promising prospect in development as drugs for relieving cough in placement of codeine phosphate or naringin in clinic.

### EMBODIMENTS

### I. Preparation of 6,8-Substituted Naringenin Derivative of the Present Invention

The preparation of the 6,8-substituted naringenin derivatives in Example 1-15 necessitates the intermediate compounds (18) and (19).

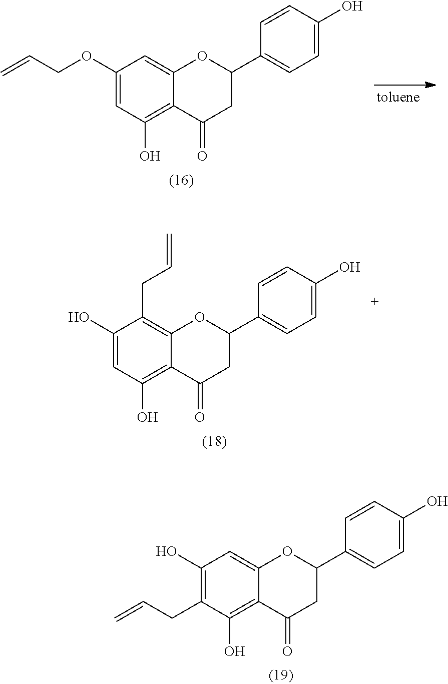

A known compound 7-allyloxynaringenin (16) as starting material was subjected to Claisen rearrangement in toluene, to obtain two isomers (18) and (19) that are 8-allylnaringenin and 6-allylnaringenin respectively.

The preparation process was as follows. The compound 7-allyloxynaringenin (16) (7.8 g) was placed in a 100 ml sealed glass reaction vessel and stood overnight in a sand bath at 180° C. The reactant was purified by silica gel column chromatography, to afford the compound (18) (1.8 g, yield 23%) and the compound (19) (1.25 g, yield 16%).

Compound (18): Mp. 190-191° C.; Thin layer chromatography (TLC) Rf=0.45 (petroleum ether:ethyl acetate 2:1). High resolution mass spectrum m/z 312.0992[M]⁺; Molecular formula C₁₈H₁₆O₅

¹H-NMR (400 MHz, DMSO-d6): 2.752 (1H,dd,J₁=17.2 Hz, J₂=3.2 Hz, H-3a), δ3.170 (1H, dd, J₁=12.4 Hz, H-3b), δ3.156 (1H, d, J=6.4 Hz,H-1″), δ4.890 (2H,m,H-3″), δ5.423 (1H,dd,J₁=12.4 Hz,J₂=2.8 Hz,H-2), δ5.801 (1H,m, H-2″), δ5.993 (1H,s, H-8), δ6.800 (1H,d,J=8.4 Hz,H-3′, H-5′), δ7.302 (1H, d, J=8.4 Hz, H-2′,H-6′), δ9.526 (1H, s,−4′OH), δ10.734 (1H,s,−7OH), δ12.113 (1H, s,−5OH).

¹³C-NMR (100 MHz, DMSO-d6): δ197.09 (C-4), δ164.86 (C-7), δ161.84 (C-5), δ160.38 (C-9), δ157.99 (C-4′), δ136.73 (C-2″), δ129.65 (C-1′), δ128.27 (C-2′,C-6′), δ115.63 (C-3′,C-5′), δ114.78 (C-3″), δ105 0.54 (C-8), δ102.23 (C-10), δ95.74 (C-6), δ78.54 (C-2), δ42.33 (C-3), δ26.73 (C-1″).

Compound (19): Mp. 193-195° C.; Thin layer chromatography (TLC) Rf=0.5 (petroleum ether:ethyl acetate 2:1). High resolution mass spectrometry m/z 312.0991[M]⁺; Molecular formula C₁₈H₁₆O₅

¹H-NMR (400 MHz, DMSO-d6): 2.681 (1H, dd, J₁=16.8 Hz, J₂=2.8 Hz, H-3a), δ3.222 (1H,dd,J₁=17.2 Hz,J₂=4.4 Hz,H-3b), δ3.182 (1H,d, J=6.4 Hz, H-1″), δ4.910 (2H, m, H-3″), δ5.412 (1H,dd,J₁=12.8 Hz, J₂=2.8 Hz, H-2), δ5.831 (1H, m, H-2″), δ5.986 (1H,s, H-6), δ6.794 (1H, d, J=8.4 Hz, H-3′,H-5′), δ7.311 (1H,d, J=8.4 Hz,H-2′,H-6′), δ9.549 (1H,s,−4′OH), δ10.732 (1H,s,−7OH), δ12.422 (1H,s,−5OH.

¹³C-NMR (100 MHz, DMSO-d6): δ196.94 (C-4), δ164.83 (C-5,C-9), δ158.16 (C-4′), δ136.56 (C-2″), δ129.42 (C-1′), δ128.72 (C-2′,C-6′), δ115.61 (C-3′,C-5′), δ114.75 (C-3″), δ106.10 (C-8), δ102.00 (C-10), δ94.79 (C-6), δ78.83 (C-2), δ42.52 (C-3) δ26.14 (C-1″).

### EXAMPLE 1

### 8-n-propylnaringenin (8-n-propyl-2,3-dihydro-5,7-dihydroxy-2-(4-hydroxyphenyl)chromen-4-one)

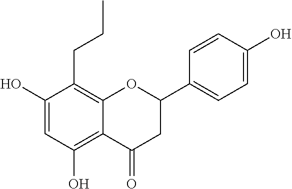 [SMILES: CCCc1c(O)cc(O)c2c1OC(c1ccc(O)cc1)CC2=O]

8-allylnaringenin (Compound 18) (1 g) was dissolved in ethanol (100 ml), and then 10% Pd/C (0.5 g) was added. The reaction system was hydrogenated for 20 hrs under normal pressure, and filtered under suction. The filtrate was concentrated and then the residue was purified by silica gel column chromatography eluting with petroleum ether:ethyl acetate=2:1, to afford a yellow solid (0.4 g). Mp. 195-197° C. Thin layer chromatography was performed with petroleum ether:ethyl acetate=1:1 as developing solvent. Rf=0.6. LRMS M-H=313.4, HPLC purity=99%, yield=40%.

¹H-NMR (400 MHz, DMSO-d6): δ0.872 (3H,t,J=7.2 Hz,H-3″), δ1.441 (2H,q, J=7.2 Hz,H-2″), δ2.415 (2H,t,J=8.4 Hz,H-1″), δ2.651 (1H,dd,J₁=16.8 Hz,J₂=2.8 Hz, H-3a), δ3.213 (1H,dd,J₁=12.8 Hz,J₂=16.8 Hz,H-3b), δ5.386 (1H,dd,J₁=12.8 Hz, J₂=2.8 Hz, H-2), δ5.945 (1H,s,H-6), δ6.788 (2H,d,J=8.8 Hz,H-3′,H-5′), δ7.301 (2H,d, J=8.8 Hz,H-2′,H-6′), δ12.416 (1H, s, 5-OH).

¹³C-NMR (100 MHz, DMSO-d6): δ196.61 (C-4), δ165.59 (C-7), δ161.31 (C-5), δ160.90 (C-9), δ158.12 (C-4′), δ129.52 (C-1′), δ128.66 (C-2′,C-6′), δ115.58 (C-3′,C-5′), δ108.67 (C-10), δ101.76 (C-8), δ94.86 (C-6), δ78.71 (C-2), δ42.48 (C-3) δ23.93 (C-2″), δ22.11 (C-1″), δ14.40 (C-3″).

### EXAMPLE 2

### 6-n-propylnaringenin (6-n-propyl-2,3-dihydro-5,7-dihydroxy-2-(4-hydroxyphenyl)chromen-4-one)

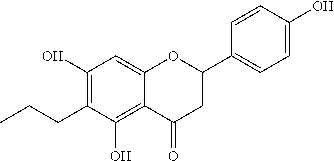 [SMILES: CCCc1c(O)cc2c(c1O)C(=O)CC(c1ccc(O)cc1)O2]

6-allylnaringenin (Compound 19) (1 g) was dissolved in ethanol (100 ml), and then 10% Pd/C (0.2 g) was added. The reaction system was hydrogenated for 20 hrs under normal pressure, and filtered under suction. The filtrate was concentrated and then the residue was purified by silica gel column chromatography eluting with petroleum ether:ethyl acetate=3:1, to afford a light yellow solid (0.4 g). Mp. 173-175° C. Thin layer chromatography was performed with petroleum ether:ethyl acetate=1:1 as developing solvent. Rf=0.6. MS M-H=313.2, HPLC purity=96%, yield=13%.

¹H-NMR (400 MHz, DMSO-d6): δ0.822 (3H, t, J=7.2 Hz; H-3″), δ1.417 (2H,q, J=7.2 Hz,H-2″), δ2.397 (2H,t,J=7.2 Hz,H-1″), δ2.733 (1H,dd,J₁=17.2 Hz, J₂=3.2 Hz, H-3a), δ3.162 (1H,dd,J₁=12.4 Hz,J₂=16.8 Hz,H-3b), δ5.408 (1H,dd,J₁=12.4 Hz, J₂=2.8 Hz,H-2), δ5.976 (1H,s,H-8), δ6.801 (2H,d,J=8.4 Hz,H-3′,H-5′), δ7.304 (2H,d, J=8.4 Hz,H-2′,H-6′), δ9.527 (1H,s,4′-OH), δ10.630 (1H,s,7-OH), δ12.112 (1H,s,5-OH).

¹³C-NMR (100 MHz, DMSO-d6): δ197.14 (C-4), δ165.07 (C-7), δ161.53 (C-5), δ160.42 (C-9), δ157.97 (C-4′), δ129.79 (C-1′), δ128.24 (C-2′,C-6′), δ115.65 (C-3′,C-5′), δ107.98 (C-10), δ102.19 (C-8), δ95.71 (C-6), δ78.45 (C-2), δ42.44 (C-3) δ24.31 (C-2″), δ22.33 (C-1″), δ14.25 (C-3″).

### EXAMPLE 3

### 8-allyl-6-bromonaringenin (8-allyl-6-bromo-2,3-dihydro-5,7-dihydroxy-2-(4-hydroxyphenyl)chromen-4-one)

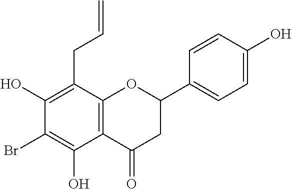 [SMILES: C=CCc1c(O)c(Br)c(O)c2c1OC(c1ccc(O)cc1)CC2=O]

8-allylnaringenin (Compound 18) (0.7 g) was dissolved in tetrachloromethane (CCl₄) (50 ml), dimethyl formamide (2 ml) was added, and then N-bromosuccinimide (NBS) (1.05 eq) was added, and heated to reflux for 5 hrs. After cooling and concentration, the residue was directly mixed with silica gel and purified by silica gel column chromatography (petroleum ether:ethyl acetate=5:1), to obtain a light yellow solid (0.48 g). Mp. 203-205° C. Thin layer chromatography was performed with petroleum ether:ethyl acetate=3:1 as developing solvent. Rf=0.6. LRMS: M-H=391.2, 389.2; HPLC purity=89.22%, yield 55%.

¹H-NMR (400 MHz, DMSO-d6): δ2.82 (1H,dd,J₁=2.8 Hz, J₂=17.2 Hz, H-3b), δ3.275 (3H,m,H-1″,H-3a), δ4.901 (2H,m,H-3″), δ5.478 (1H,dd,J₁=2.8 Hz, J₂=12.8 Hz, H-2), δ5.795 (1H,m,H-2″), δ6.800 (2H,t,H-3′,H5′), δ7.305 (2H,d,J=8.8 Hz,H-2′,H-6′), δ9.553 (1H,s,H-4′OH), δ10.334 (1H,s,H-7OH), δ12.840 (1H,s,H-5OH).

¹³C-NMR (100 MHz, DMSO-d6): δ197.59 (C-4), δ160.59 (C-5), δ159.33 (C-7), δ158.16 (C-9), δ158.10 (C-4′), δ136.15 (C-2″), δ129.25 (C-1′), δ128.36 (C-2′,C-6′), δ115.66 (C-3′,C-5′), δ115.16 (C-3″), δ107.01 (C-8), δ102.91 (C-10), δ90.84 (C-6), δ78.77 (C-2), δ42.03 (C-3), δ27.32 (C-1″).

### EXAMPLE 4

### 6-allyl-8-bromonaringenin (6-allyl-8-bromo-2,3-dihydro-5,7-dihydroxy-2-(4-hydroxyphenyl)chromen-4-one)

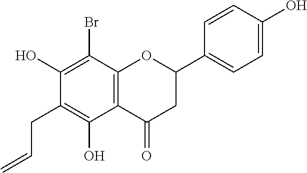 [SMILES: C=CCc1c(O)c(Br)c2c(c1O)C(=O)CC(c1ccc(O)cc1)O2]

6-allylnaringenin (Compound 19) (0.7 g) was dissolved in tetrachloromethane (50 ml), dimethyl formamide (2 ml) was added, and then N-bromosuccinimide (NBS) (1.05 eq) was added, and heated to reflux for 5 hrs until the raw materials were found to disappear as indicated by thin layer chromatography (TLC). After cooling and concentration, the residue was directly mixed with silica gel and purified by silica gel column chromatography (petroleum ether:ethyl acetate=5:1), to obtain a light yellow solid (0.7 g). Mp. 158-160° C. Thin layer chromatography was performed with petroleum ether:ethyl acetate=2:1 as developing solvent. Rf=0.6. LRMS: M-H=391.2, 389.2; HPLC purity=96.9%, yield 89%.

¹H-NMR (400 MHz, DMSO-d6): δ2.843 (1H,ddA=3.2 Hz,J₂=17.2 Hz,H-3b), δ3.342 (3H,m,H-1″,H-3a), δ4.946 (2H,m,H-3″), δ5.589 (1H,dd,J₁=2.8 Hz,J₂=12.6 Hz,H-2), δ5.840 (1H,m,H-2″), δ6.814 (2H,t,H-3 ‘,HS’), δ7.344 (2H,d,J=8.8 Hz,H-2′,H-6′), δ9.584 (1H,s,H-4′OH), δ10.297 (1H,s,H-7OH), δ12.458 (1H,s,H-5OH).

¹³C-NMR (100 MHz, DMSO-d6): δ197.35 (C-4), δ160.63 (C-5), δ160.05 (C-7), δ158.21 (C-9), δ157.42 (C-4′), δ135.98 (C-2″), δ128.97 (C-1′), δ128.59 (C-2′,C-6′), δ115.68 (C-3′,C-5′), δ115.12 (C-3″), δ107.62 (C-6), δ103.01 (C-10), δ89.77 (C-8), δ79.38 (C-2), δ41.99 (C-3), δ26.70 (C-1″).

### EXAMPLE 5

### 6-allyl-8-chloronaringenin (6-allyl-8-chloro-2,3-dihydro-5,7-dihydroxy-2-(4-hydroxyphenyl)chromen-4-one)

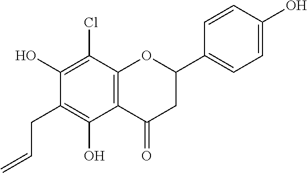 [SMILES: C=CCc1c(O)c(Cl)c2c(c1O)C(=O)CC(c1ccc(O)cc1)O2]

6-allylnaringenin (Compound 19) (0.5 g) was dissolved in tetrachloromethane (50 ml), dimethyl formamide (2 ml) was added, and then N-chlorosuccinimide (NBS) (1.05 eq) was added, and heated to reflux for 5 hrs. After cooling and concentration, the residue was directly mixed with silica gel and purified by silica gel column chromatography (petroleum ether:ethyl acetate=5:1), to obtain a light yellow solid (0.3 g). Mp. 158-160° C. Thin layer chromatography was performed with petroleum ether:ethyl acetate=3:1 as developing solvent. Rf=0.6. LRMS: M-H=346.2; HPLC purity=95.5%, yield 54%.

¹H-NMR (400 MHz, DMSO-d6): δ2.814 (1H,ddA=2.8 Hz,J₂=17.4 Hz,H-3b), δ3.342 (3H,m,H-1″,H-3a), δ4.947 (2H,q,H-3″), δ5.584 (1H,dd,J₁=2.8 Hz,J₂=12.6 Hz, H-2), δ5.850 (1H,m,H-2″), δ6.820 (2H,d, J=8.4 Hz,H-3′,H5′), δ7.344 (2H,d,J=8.4 Hz, H-2′,H-6′), δ9.600 (1H,s, H-4′OH), δ10.502 (1H,s,H-7OH), δ12.393 (1H,s,H-5OH).

¹³C-NMR (100 MHz, DMSO-d6): δ197.36 (C-4), δ159.81 (C-5), δ159.27 (C-7), δ158.31 (C-9), δ156.35 (C-4′), δ136.02 (C-2″), δ128.92 (C-1′), δ128.75 (C-2′,C-6′), δ115.72 (C-3′,C-5′), δ115.16 (C-3″), δ107.56 (C-8), δ102.73 (C-10), δ99.60 (C-6), δ79.57 (C-2), δ42.09 (C-3), δ26.62 (C-1″).

### EXAMPLE 6

### 8-propyl-6-chloronaringenin(6-chloro-2,3-dihydro-5,7-dihydroxy-2-(4-hydroxyphenyl)-8-propylchromen-4-one)

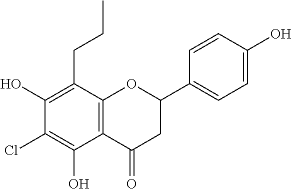 [SMILES: CCCc1c(O)c(Cl)c(O)c2c1OC(c1ccc(O)cc1)CC2=O]

8-n-propylnaringenin (Example 1) (0.1 g) was dissolved in methanol (MeOH) (100 ml), and then 10% Pd/C (0.1 g) was added. The reaction system was evacuated, and then hydrogen was introduced, reacted for 3 hrs under 1 atm, and filtered. The filtrate was concentrated and then the residue was purified by silica gel chromatography, to obtain a light yellow solid (76 mg). Mp. 175-176° C. Thin layer chromatography was performed with petroleum ether:ethyl acetate=3:1 as developing agent. Rf=0.55. LRMS: M-H=347.3, 349.4, HPLC purity=98.5%, yield 75%.

¹H-NMR (400 MHz, DMSO-d6): δ0.893 (3H,t,H-3″), δ1.457 (2H,q,H-2″), δ2.523 (2H,m,H-1″), δ2.802 (1H,dd,J₁=2.8 Hz,J₂=17.2 Hz, H-3b), δ3.351 (1H,q,H-3a), δ5.570 (1H,dd,J₁=2.8 Hz,J₂=12.8 Hz,H-2), δ6.818 (2H, t,H-3′,HS′), δ7.340 (2H,d,J=8.4 Hz, H-2′, H-6′), δ9.591 (1H,s,H-4′OH), δ10.366 (1H,s,H-7OH), δ12.386 (1H,s,H-5OH).

¹³C-NMR (100 MHz, DMSO-d6): δ197.33 (C-4), δ159.82 (C-5), δ159.33 (C-4′), δ158.27 (C-9), δ155.96 (C-7), δ128.95 (C-1′), δ128.71 (C-2′,C-6′), δ115.68 (C-3′,C-5′), δ110.07 (C-10), δ102.67 (C-8), δ99.53 (C-6), δ79.46 (C-2), δ42.11 (C-3), δ24.43 (C-2″), δ22.05 (C-1″), δ14.28 (C-3″).

### EXAMPLE 7

### 8-allyl-6-chloronaringenin (8-allyl-6-chloro-2,3-dihydro-5,7-dihydroxy-2-(4-hydroxyphenyl)chromen-4-one)

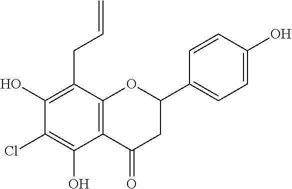 [SMILES: C=CCc1c(O)c(Cl)c(O)c2c1OC(c1ccc(O)cc1)CC2=O]

8-allylnaringenin (Compound 18) (0.5 g) was dissolved in tetrachloromethane (50 ml), dimethyl formamide (2 ml) was added, and then N-chlorosuccinimide (NCS) (1.05 eq) was added and heated to reflux for 5 hrs. After cooling and concentration, the residue was directly mixed with silica gel and purified by silica gel column chromatography (petroleum ether:ethyl acetate=5:1), to obtain a light yellow solid (0.15 g). Mp. 215-216° C. Thin layer chromatography was performed with petroleum ether:ethyl acetate=3:1 as developing solvent. Rf=0.6. LRMS: M-H=346.2, HPLC purity=94.2%, yield 27%.

¹H-NMR (400 MHz, DMSO-d6): δ2.831 (1H,ddA=2.8 Hz,J₂=17.2 Hz,H-3b), δ3.283 (3H,m,H-1″,H-3a), δ4.908 (2H,m,H-3″), δ5.483 (1H, dd,J₁=2.8 Hz,J₂=12.8 Hz, H-2), δ5.810 (1H,m,H-2″), δ6.804 (2H,t,H-3′, H5 ‘), δ7.311 (2H,d,J=8.8 Hz,H-2’,H-6′), δ9.562 (1H,s,H-4′OH), δ10.553 (1H,s,H-7OH), δ12.713 (1H,s,H-5OH).

¹³C-NMR (100 MHz, DMSO-d6): δ197.74 (C-4), δ159.70 (C-5), δ158.55 (C-7), δ158.10 (C-9), δ157.00 (C-4′), δ136.15 (C-2″), δ129.26 (C-1′), δ128.36 (C-2′,C-6′), δ115.66 (C-3′,C-5′), δ115.17 (C-3″), δ107.01 (C-8), δ102.63 (C-10), δ100.45 (C-6), δ78.79 (C-2), δ42.15 (C-3) δ27.18 (C-1″).

### EXAMPLE 8

### 6-allyl-8-hydroxynaringenin(6-allyl-2,3-dihydro-5,7,8-Trihydroxy-2-(4-hydroxyphenyl)chromen-4-one)

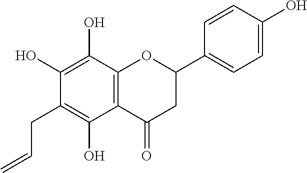 [SMILES: C=CCc1c(O)c(O)c2c(c1O)C(=O)CC(c1ccc(O)cc1)O2]

6-bromo-8-allylnaringenin (Example 3) (9 g) was dissolved in ethyl acetate (250 ml), and then m-chloroperoxybenzoic acid (10 g) was added, heated to reflux for 48, and filtered under suction. The filtrate was concentrated and then the residue was purified by silica gel column chromatography, to obtain a light yellow solid (280 mg). Mp. 95-100° C. Thin layer chromatography was performed with petroleum ether:ethyl acetate=1:1 as developing solvent. Rf=0.6. LRMS: M-H=327.3, HPLC purity=86.5%, yield 29.6%.

¹H-NMR (400 MHz, DMSO-d6): δ2.732 (1H,dd,J₁=2.8 Hz,J₂=17.2 Hz,H-3b), δ3.259 (3H,m,H-1″,H-3a), δ4.921 (2H,q,H-3″), δ5.436 (1H,dd,J₁=2.8 Hz,J₂=12.0 Hz,H-2), δ5.844 (1H,d,J=6.8 Hz,H-2″), δ6.805 2H,ddA=4.0 Hz,J₂=8.4 Hz,H-3′,HS′), δ7.377 (2H,d,J=8.8 Hz,H-2′, H-6′), δ8.368 (1H,s,H-8OH), δ9.568 (1H,s,H-4′OH), δ9.877 (1H,s, H-7OH), δ12.070 (1H,s,H-5OH).

¹³C-NMR (100 MHz, DMSO-d6): δ197.07 (C-4), δ158.09 (C-4″), δ155.43 (C-5), δ154.26 (C-7), δ148.19 (C-9), δ136.61 (C-2″), δ129.60 (C-1′), δ128.77 (C-2′,C-6′), δ125.17 (C-8), δ115.52 (C-3 ‘,C-5’), δ114.73 (C-3″), δ105.86 (C-10), δ101.60 (C-6), δ78.94 (C-2), δ42.83 (C-3), δ26.47 (C-1″).

### EXAMPLE 9

### 6-propyl-8-chloronaringenin(8-Chloro-2,3-dihydro-5,7-dihydroxy-2-(4-hydroxyphenyl)-6-propylchromen-4-one)

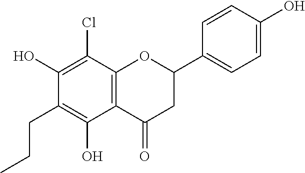 [SMILES: CCCc1c(O)c(Cl)c2c(c1O)C(=O)CC(c1ccc(O)cc1)O2]

6-n-propylnaringenin (Example 2) (0.5 g) was dissolved in tetrachloromethane (50 ml), dimethyl formamide (2 ml) was added, and then N-chlorosuccinimide (NCS) (1.05 eq) was added and heated to reflux for 5 hrs. After cooling and concentration, the residue was directly mixed with silica gel and purified by silica gel column chromatography (petroleum ether:ethyl acetate=5:1), to obtain a yellow solid (0.1 g). Mp. 175-176° C. Thin layer chromatography was performed with petroleum ether:ethyl acetate=3:1 as developing solvent. Rf=0.6. LRMS: m/z 348.0759[M]⁺.

¹H-NMR (400 MHz, DMSO-d6): δ0.838 (3H,t=7.2 Hz,H-3″), δ1.435 (2H,ddA=7.2 Hz,J₂=14.8 Hz,H-2″), δ2.498 (2H,m,H-1″), δ2.816 (1H,dd,J₁=2.8 Hz,J₂=17.4 Hz,H-3b), δ3.285 (1H,m,H-3a), δ5.469 (1H,dd,J₁=2.8 Hz,J₂=12.8 Hz,H-2), δ6.814 (2H,d,J=8.8 Hz,H-3′,HS′), δ7.315 (2H,d,J=8.4 Hz,H-2′,H-6′), δ9.594 (1H,s,H-4′OH), δ10.422 (1H,s, H-7OH), δ12.708 (1H,s,H-5OH).

¹³C-NMR (100 MHz, DMSO-d6): δ197.81 (C-4), δ159.80 (C-5), δ158.61 (C-4′), δ158.09 (C-7), δ156.68 (C-9), δ129.40 (C-1′), δ128.32 (C-2′,C-6′), δ115.69 (C-3′,C-5′), δ109.47 (C-6), δ102.60 (C-10), δ100.42 (C-8), δ78.70 (C-2), δ42.26 (C-3), δ24.84 (C-1″), δ22.27 (C-2″), δ14.14 (C-3″).

### EXAMPLE 10

### 6-allyl-8-iodonaringenin (8-iodo-2,3-dihydro-5,7-dihydroxy-2-(4-hydroxyphenyl)-6-allylchromen-4-one)

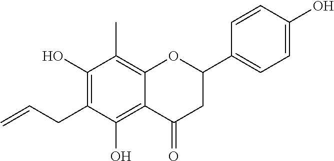 [SMILES: C=CCc1c(O)c(C)c2c(c1O)C(=O)CC(c1ccc(O)cc1)O2]

6-allylnaringenin (Compound 19) (0.5 g) was dissolved in tetrachloromethane (50 ml), DMF (2 ml) was added, and then N-iodosuccinimide (NIS) (1.05 eq) was added and heated to reflux for 3 hrs. The sample was directly mixed with silica gel and purified by silica gel column chromatography (petroleum ether:ethyl acetate=5:1), to obtain a light yellow solid (0.18 g). Mp. 218-220° C. Thin layer chromatography was performed with petroleum ether:ethyl acetate=3:1 as developing solvent. Rf=0.6. LRMS: M-H=437.2, HPLC purity=93.79%, yield 25%.

¹H-NMR (400 MHz, DMSO-d6): δ(2H,m,H-3b), δ3.330 (3H,m,H-1″,H-3a), δ6.817 (2H,dd,J₁=1.6 Hz,J₂=8.4 Hz,H-3′,H5′), δ7.350 (2H,q,H-2′,H-6′), δ9.579 (1H,s,H-4′OH), δ12.286 (1H,s,H-5OH).

### EXAMPLE 11

### Naringenin-8-acetaldehyde

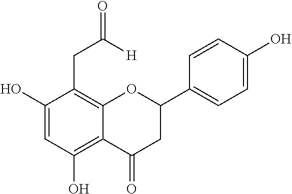 [SMILES: O=CCc1c(O)cc(O)c2c1OC(c1ccc(O)cc1)CC2=O]

8-allylnaringenin (Compound 18) (2 g) was dissolved in dichloromethane (100 ml), and then tetrahydrofuran (THF) (20 ml) was added. The reaction vessel was evacuated, and then nitrogen was introduced. The solution was cooled to −70° C. in a dry ice-acetone bath, then ozone was continuously introduced for about 2 hrs until the solution becomes blue colored and no discoloration occurs in ten minutes after the introduction of ozone is complete. Whereupon air was introduced for 15 min, then a reducing agent dimethyl sulfide (15 ml) was added and gradually heated to room temperature. The sample was mixed with silica gel and purified by silica gel column chromatography, to obtain a liquid (1 g), which was recrystallized in dichloromethane, to obtain a light yellow solid (0.3 g). Mp. 150-155° C. Thin layer chromatography was performed with petroleum ether:ethyl acetate=1:1 as developing solvent. Rf=0.5. LRMS: M-H=313.3, yield 15%.

¹H-NMR (400 MHz, DMSO-d6): δ2.701 (1H, m), δ3.243 (1H, m), δ3.482 (2H,s), δ5.450 (1H, m), δ6.807 (2H, d, J=8.4 Hz), δ7.319 (1H, d, J=8.4 Hz), δ9.540, δ9.572 (−4′OH), δ11.034 (−7OH), δ12.440 (−5OH).

¹³C-NMR (100 MHz, DMSO-d6): δ200.531, δ196.872, δ165.146, δ164.563, δ162.621, δ158.244, δ128.761, δ128.224, δ115.677, δ104.383, δ94.757, δ8.895, δ42.396, δ37.748, δ33.703.

### EXAMPLE 12

### Naringenin-6-acetaldehyde

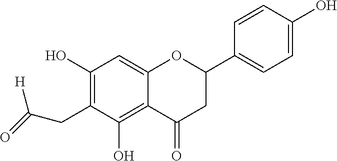 [SMILES: O=CCc1c(O)cc2c(c1O)C(=O)CC(c1ccc(O)cc1)O2]

6-allylnaringenin (Compound 19) (12 g) was dissolved in ethyl acetate (100 ml). The reaction vessel was evacuated, and then nitrogen was introduced. The solution was cooled to −70° C. in a dry ice-acetone bath, then ozone was continuously introduced for about 2 hrs until the solution becomes blue colored and no discoloration occurs in ten minutes after the introduction of ozone is complete. Whereupon air was introduced for 15 min, then a reducing agent dimethyl sulfide (15 ml) was added, gradually heated to room temperature and stirred for 2 hrs. The sample was mixed with silica gel and purified by silica gel column chromatography, to obtain a yellow solid (4.5 g). Mp. 178-180° C. Thin layer chromatography was performed with petroleum ether:ethyl acetate=1:1 as developing solvent. Rf=0.5. LRMS: M-H=313.3, yield 37%.

¹H-NMR (400 MHz, DMSO-d6): δ2.708 (1H, m), δ3.243 (1H, m), δ3.514 (2H,s), δ5.470 (1H, m), δ6.799 (2H, d, J=8.4 Hz), δ7.321 (1H, d, J=8.4 Hz), δ9.573 (−4′OH), δ11.028 (−7OH), 12.489 (−5OH).

¹³C-NMR (100 MHz, DMSO-d6): δ200.447, δ197.059, δ165.164, δ162.067, δ161.735, δ158.221, δ129.314, δ128.777, δ115.651, δ100.310, δ4.809, δ9.158, δ42.412, δ7.234, δ3.369.

### EXAMPLE 13

### 6-chloro-naringenin-8-acetic acid

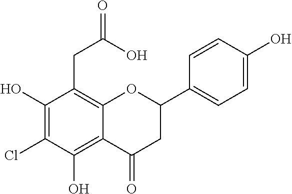 [SMILES: O=C(O)Cc1c(O)c(Cl)c(O)c2c1OC(c1ccc(O)cc1)CC2=O]

Naringenin-8-acetaldehyde (Example 11) (0.2 g) was dissolved in ethanol (100 ml), aminosulfonic acid (1.1 eq) was added, and then sodium chlorite (1.1 eq) was added dropwise at freezing point. The reaction was completed in 0.5 hr. The sample was mixed with silica gel and purified by silica gel column chromatography, to obtain a light yellow solid (0.05 g). Mp. 185-186° C. Thin layer chromatography was performed with dichloromethane:methanol=5:1 as developing solvent. Rf=0.5. LRMS: M-H=363.2, 365.2, HPLC purity=92.5%, yield 20%.

¹H-NMR (400 MHz, DMSO-d6): δ2.868 (1H, m), δ3.271 (1H, m), δ3.451 (2H, m), δ5.493 (1H, m), δ6.809 (2H, d, J=8.4 Hz), δ7.308 (1H,d,J=8.4 Hz), δ9.561 (−4′OH), δ10.784 (−7OH), δ12.725 (−5OH).

¹³C-NMR (100 MHz, DMSO-d6): δ197.624, δ172.381, δ160.160, δ158.870, δ158.135, δ157.478, δ129.116, δ128.378, δ115.680, δ103.754, δ102.513, δ100.419, δ78.941, δ42.124, δ29.107.

### EXAMPLE 14

### 8-chloro-naringenin-6-acetic acid

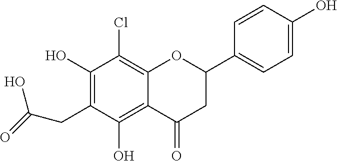 [SMILES: O=C(O)Cc1c(O)c(Cl)c2c(c1O)C(=O)CC(c1ccc(O)cc1)O2]

Naringenin-6-acetaldehyde (Example 12) (0.7 g) was dissolved in ethyl acetate (100 ml), aminosulfonic acid (1.1 eq) was added, and then sodium chlorite (1.1 eq) was added dropwise at freezing point. The reaction was completed in 0.5 hr. The sample was mixed with silica gel and purified by silica gel column chromatography, to obtain a light yellow solid (0.2 g). Mp. 185-186° C. Thin layer chromatography was performed with dichloromethane:methanol=5:1 as developing solvent. Rf=0.5. LRMS: M-H=363.2, 365.2, HPLC purity=97.34%, yield 24.5%.

¹H-NMR (400 MHz, DMSO-d6): δ12.395 (1″-OH), δ7.357 (2′,6′-H, 2H,d, J=8.5 Hz), δ6.831 (3′,5′-H,2H,d,J=8.5 Hz), δ5.614 (2-H,1H,dd,J=12.5 Hz,J=3.0 Hz) δ4.044 (3-H, H, m, J=13.5 Hz,J=7.0 Hz), 83.478 (2″-H, 2H, S), δ2.844 (3-H,H,dd, J=17.0 Hz,J=3.0 Hz).

¹³C-NMR (100 MHz, DMSO-d6): δ197.285, δ172.380, δ160.245, δ159.502, δ158.328, δ156.780, δ128.878, δ128.746, δ115.737, δ104.299, δ102.581, δ99.537, δ79.620, δ42.014, δ28.487.

### EXAMPLE 15

### Naringenin-8-acetic acid

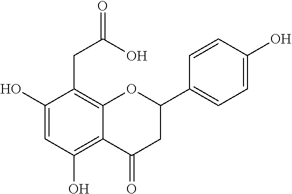 [SMILES: O=C(O)Cc1c(O)cc(O)c2c1OC(c1ccc(O)cc1)CC2=O]

Naringenin-8-acetaldehyde (Example 11) (1 g) was dissolved in ethyl acetate, aminosulfonic acid (1.1 eq) was added, and then sodium chlorite (1.1 eq) was added dropwise at freezing point. The reaction was completed in 0.5 hr. The sample was mixed with silica gel and purified by silica gel column chromatography, to obtain a light yellow solid (0.2 g). Mp. 245-247° C. Thin layer chromatography was performed with dichloromethane:methanol=5:1 as developing solvent. Rf=0.5. LRMS: M-H=329.4, yield 19%.

¹H-NMR (400 MHz, DMSO-d6): δ2.703 (1H, m), δ3.271 (1H, m), δ3.709 (2H,s), δ5.416 (1H, m), δ6.797 (2H, d, J=8.4 Hz), δ7.324 (1H, d, J=8.4 Hz), δ9.560 (−4′OH), δ12.760 (−5OH).

### II. Test on Cough Relieving Effect of Singly Administered Example Compounds of the Present Invention in Mice with Cough Induced from Aqueous Ammonia

(i) Materials:

1. Test animals: male KM mice (available from Guangdong Medical Laboratory Animal Center, Certificate No.: SCXK (Guangdong) 2008-0002, No. 0113738).

2. Test groups and treatment with drugs: Blank control group: treatment with 50% (v/v) PEG400 in physiological saline.

Treatment group with codeine phosphate as positive control—Codeine phosphate (Lott 20091212) is manufactured by Qinghai Pharmaceutical Co., Ltd. Before test, powdered codeine phosphate was added to 50% (v/v) PEG400 in physiological saline, to formulate a 0.47 μmol/ml solution.

Example compounds: the powdered example compounds were formulated into 50% (v/v) PEG400 in physiological saline respectively, to give a 7.44 μmol/ml solution or suspension.

3. Reagents and Instruments:

YLS-8A cough and wheeze inducing instrument (a product available from Equipment Station, Shandong Academy of Medical Science).

Codeine phosphate (Qinghai Pharmaceutical Co., Ltd, Lott 20091212), 25% aqueous ammonia (Tianjin Guangcheng Chemical Reagent Co. Ltd., Lott 20110515), PEG400 (Tianjin Guangcheng Chemical Reagent Co. Ltd., Lott 20110308), 0.9% sodium chloride injection (Guangzhou Kelun Pharmaceutical Co. Ltd., Lott D1201104-1). Intragastric needle: customized in Laburatory Animal Center, Guangdong Test Center for Occupational Health.

(ii) Test environment

Laboratory Animal Center, Sun Yat-sen University.

(iii) Test method

Eligible male KM mice were randomly designated to a blank control group, and treatment groups with codeine phosphate as positive control and with example compounds, according to tablet.

The animals in each group were administered by intragastric gavage or intraperitoneal injection at a dosage of 0.2 ml/10 g of body weight, and placed in a cough inducing chamber 1 hr after administration. 1 ml of 25% concentrated aqueous ammonia was added to an atomizing cup and atomized for 15 s (at a rate of 0.15 ml/min). The coughs in 3 min were counted (during the typical cough, the mouth was opened and the head was lowered, which were accompanied by quivering of the neck and frequently a forward rushing).

Statistical analysis of data—Statistical analysis of all the data was conducted by SPSS 17.0 after the outliers were excluded by t test. Analysis of variance was performed by one-way ANOVA, multiple comparisons between the blank control group and each treatment group were carried out by Dunnett test, and the pairwise multiple comparisons between each treatment group and the codeine phosphate group were conducted by Duncan test.

(iv) Test results—After statistical analysis, coughs of the animals in each group in 3 min after atomization are shown in Table 1.

TABLE 1
Cough relieving effect of each compound in mice with cough
induced from aqueous ammonia

| | | Cough | P value compared |
|---|---|---|---|
| Groups | Coughs | relieving rate | with blank group |
| Example 1 | 6.2 ± 3.5** | 65.11% | 0.000 |
| Example 2 | 12.4 ± 4.6* | 30.16% | 0.028 |
| Example 3 | 11.2 ± 6.2* | 37.28% | 0.019 |
| Example 4 | 11.0 ± 4.1** | 38.03% | 0.005 |
| Example 5 | 11.8 ± 7.3* | 33.49% | 0.047 |
| Example 6 | 9.1 ± 3.9** | 49.06% | 0.001 |
| Example 7 | 10.5 ± 5.8** | 40.85% | 0.009 |
| Example 8 | 10.2 ± 2.6** | 42.64% | 0.002 |
| Example 9 | 10.7 ± 4.2** | 39.82% | 0.004 |
| Example 10 | 12.2 ± 4.0* | 31.24% | 0.016 |
| Example 11 | 11.4 ± 6.5* | 35.78% | 0.026 |
| Example 12 | 9.3 ± 4.3* | 47.59% | 0.002 |
| Example 13 | 7.4 ± 3.4** | 58.43% | 0.000 |
| Example 14 | 9.3 ± 3.9** | 47.59% | 0.001 |
| Example 15 | 8.3 ± 5.8** | 53.31% | 0.002 |
| codeine | 10.8 ± 3.5** | 39.60% | 0.003 |
| phosphate | | | |
| Blank group | 17.8 ± 5.4 | | |

Hereinafter, the Preparation Examples provide the pharmaceu Tical Preparations containing the 6,8-substituted naringenin derivative obtained in Example 1 of the present invention.

### Preparation Example 1

### Tablet

| | |
|---|---|
| Compound of Example 1 | 10 mg |
| Lactose | 187 mg |
| Corn starch | 50 mg |
| Magnesium stearate | 3 mg |

Preparation method—The compound of Example 1, lactose, and corn starch were mixed, uniformly wetted with water, sieved, dried, and sieved again. Magnesium stearate was added, and then the mixture was tableted. Each tablet weighed 250 mg.

### Preparation Example 2

### Capsule

| | |
|---|---|
| Compound of Example 1 | 10 mg |
| Lactose | 188 mg |
| Magnesium stearate | 2 mg |

Preparation method—The compound of Example 1, lactose, and magnesium stearate were mixed, sieved, and uniformly mixed in a suitable container, and then the mixture was filled in hard gelatin capsules. Each capsule weighed 200 mg.

### Preparation Example 3

### Ampoule

| | |
|---|---|
| Compound of Example 1 | 2 mg |
| Sodium chloride | 9 mg |

Preparation method—The Compound of Example 1 and sodium chloride were dissolved in a suitable amount of water for injection. The resulting solution was filtered and aseptically packaged in an ampoule.

### Preparation Example 4

### Nasal Spray

| | |
|---|---|
| Compound of Example 1 | 80 mg |
| Sodium chloride | 8 mg |
| EDTA | 1 mg |
| Sodium phosphate buffer (pH 6.5) | 10 mg |
| Polysorbate | 10 mg |
| Double distilled water | 2 ml |

Preparation method—One ingredient was added to a suitable amount of double distilled water at a time with stirring until it was completely dissolved, followed by the addition of another ingredient. Water was added to give a volume of 2 ml, and then the solution was filtered through an aseptic filter, filled in a vial and spaced based on a suitable dose.

1. A 6,8-substituted naringenin derivative as shown in general formula (1): 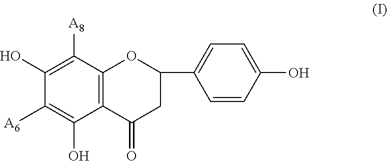
wherein A6 and A8 are respectively selected from H, halogen, hydroxyl, an alkyl group with 1-3 carbon atoms, and a straight-chain alkenyl group, alcohol group, aldehyde group or carboxylic acid group containing 2-3 carbon atoms; or a pharmaceutically acceptable salt thereof.

2. The derivative according to claim 1, wherein the alkyl group with 1-3 carbon atoms is methyl, ethyl or propyl.

3. The derivative according to claim 1, wherein the straight-chain alkenyl group, alcohol group, aldehyde group or carboxylic acid group containing 2-3 carbon atoms is straight-chain allyl, ethanol group, acetaldehyde group, or acetic acid group.

4. The derivative according to claim 1, wherein the halogen is selected from F, CI, Br or I.

5. The derivative according to claim 1, wherein the derivative is one of 8-n-propylnaringenin, 6-n-propylnaringenin, 6-bromo-8-allylnaringenin, 6-allyl-8-bromonaringenin, 6-allyl-8-chloronaringenin, 8-propyl-6-chloronaringenin, 6-chloro-8-allylnaringenin, 6-allyl-8-hydroxynaringenin, 6-propyl-8-chloronaringenin, 6-allyl-8-iodonaringenin, naringenin-8-acetaldehyde, naringenin-6-acetaldehyde, naringenin-8-acetic acid, 8-chloro-naringenin-6-acetic acid, or 6-chloro-naringenin-8-acetic acid.

6-7. (canceled)

8. A pharmaceutical preparation with the derivative according to claim 1, or a pharmaceutically acceptable salt thereof single or in combination as active ingredient(s).

9. The pharmaceutical preparation according to claim 8, wherein the pharmaceutical preparation is in a pharmaceutically acceptable dosage form, and the active ingredient is present in an amount of 0.1-95% by weight of the pharmaceutical preparation.

10. A method for preparing the derivative according to claim 1, comprising:
(1) preparing the intermediate compounds 8-allylnaringenin and 6-allylnaringenin of the derivative, specifically by subjecting a known compound 7-allyloxynaringenin as starting material to Claisen rearrangement in toluene, to obtain two isomers that are 8-allylnaringenin and 6-allylnaringenin respectively;
(2) as required by the position of the modifying group to be introduced, dissolving the intermediate compound 8-allylnaringenin or 6-allylnaringenin obtained in step (1) in ethanol, adding a catalytic amount of 10% Pd/C, hydrogenating for 20 hrs under normal pressure, filtering under suction, and purifying the filtrate by silica gel column chromatography eluting with a mixed solvent of petroleum ether and ethyl acetate, to obtain 8-n-propylnaringenin and 6-n-propylnaringenin respectively;
(3) as required by the position of the modifying group to be introduced, dissolving the intermediate compound 8-allylnaringenin or 6-allylnaringenin obtained in step (1) in tetrachloromethane and dimethyl formamide, halogenating with N-halosuccinimide, and after the reaction is complete, purifying the reaction solution by silica gel column chromatography eluting with a mixed solvent of petroleum ether and ethyl acetate, to obtain an allylnaringenin derivative substituted with halogen at position 6 or 8 respectively;
(4) as required by the position of the modifying group to be introduced, dissolving the derivative substituted with a propyl group at position 8 or 6 obtained in step (2) in tetrachloromethane and dimethyl formamide, halogenating with equivalent amount of N-halosuccinimide, and after the reaction is complete, purifying the reaction solution by silica gel column chromatography eluting with a mixed solvent of petroleum ether and ethyl acetate, to obtain a propylnaringenin derivative substituted with halogen at position 6 or 8 respectively;
(5) dissolving the intermediate compound 8-allylnaringenin or 6-allylnaringenin obtained in step (1) in dichloromethane and tetrahydrofuran, cooling the solution to −70° C. in a dry ice-acetone bath under neat nitrogen atmosphere, then introducing ozone continuously for 2 hrs until the solution becomes blue colored and no discoloration occurs in ten minutes after the introduction of ozone is complete, whereupon introducing air for 15 min, then adding a reducing agent dimethyl sulfide, gradually heating to room temperature, mixing the resulting product with silica gel and purifying by silica gel column chromatography, to obtain a naringenin derivative substituted with an aldehyde group at position 8 or 6 respectively;
(6) dissolving the allylnaringenin derivative substituted with halogen at position 6 or 8 obtained in step (3) in ethyl acetate, then adding m-chloroperoxybenzoic acid, heating to reflux for 48 hrs, filtering under suction, concentrating the filtrate, and then purifying the residue by silica gel column chromatography, to obtain an allylnaringenin derivative substituted with a hydroxyl group at position 6 or 8 respectively;
(7) dissolving the naringenin derivative substituted with an aldehyde group at position 8 obtained in step (5) in ethanol, adding aminosulfonic acid, adding sodium chlorite dropwise at freezing point, and after complete reaction, mixing the resulting product with silica gel and purifying by silica gel column chromatography, to obtain a naringenin derivative substituted with chloro at position 6 and with a carboxylic acid group at position 8, and
dissolving the naringenin derivative substituted with an aldehyde group at position 6 obtained in step (5) in ethyl acetate, adding aminosulfonic acid, adding sodium chlorite dropwise at freezing point, and after complete reaction, mixing the resulting product with silica gel and purifying by silica gel column chromatography, to obtain a naringenin derivative substituted with chloro at position 8 and with a carboxylic acid group at position 6; and
(8) dissolving the naringenin derivative substituted with an aldehyde group at position 8 obtained in step (5) in ethyl acetate, adding aminosulfonic acid, adding sodium chlorite dropwise at freezing point, and after the reaction is completed in 0.5 hr, mixing the sample with silica gel and purifying by silica gel column chromatography, to obtain a naringenin derivative substituted with H at position 6 and with a carboxylic acid group at position 8.
